(19) United States
(12) Patent Application Publication
(10) Pub. No.: US 2013/0172899 A1
(43) Pub. Date: Jul. 4, 2013

(54) Devices For Cast Removal, Methods of Applying a Cast and Removal Device to a Patient, and Methods of their Removal

(71) Applicant: Olarewaju Oladipo, Canton, MA (US)

(72) Inventor: Olarewaju Oladipo, Canton, MA (US)

(21) Appl. No.: 13/541,989

(22) Filed: Jul. 5, 2012

Related U.S. Application Data
(63) Continuation-in-part of application No. 13/342,054, filed on Jan. 1, 2012.
(60) Provisional application No. 61/616,113, filed on Mar. 27, 2012.

(51) Int. Cl. A61F 5/04

(52) U.S. Cl. 606/105.5

(57) ABSTRACT

A cast removal device having an elongated cord with at least two free ends and a housing, having an elongated trough, methods of applying the cast removal device to a body member of a patient and methods of removing the cast.

### RELATED APPLICATIONS

This application is a continuation-in-part application of and claims priority to and benefit of U.S. patent application Ser. No. 13/342,054, filed on Jan. 1, 2012, and claims priority to and benefit of U.S. Provisional Application 61/616,113, filed on Mar. 27, 2012, the entire contents of each are incorporated by reference in their entirety.

### TECHNICAL FIELD OF THE INVENTION

The present invention relates to the care of orthopedic conditions, particularly injuries requiring treatment with casting materials, a kit or device for removing casting materials from the patient, and methods thereof.

### BACKGROUND OF THE INVENTION

Treatment of orthopedic injuries often requires the use of casting materials. Removal of an orthopedic cast is necessary at some point in the care of an injured patient. Current convention dictates that an applied orthopedic cast (“cast”, hereinafter) is removed with a shear, or an electric saw.

The use of shears to remove an applied cast is limited to remote settings where access to battery or electric power is a challenge. The use of a shear during cast removal is less than ideal because its lower blade is pushed against the skin of the injured part of the body, causing unnecessary pressure, pain and discomfort.

The use of a traditional cast saw, though efficient, is limited to settings where an electricity source, or battery power is readily available. The use of a cast saw causes noise, and dust pollution from the dust that fills the air during cast removal. The noisy traditional cast saw may have the undesirable effect of generating fear in children, or even in adults who find the use of a cast saw on an extremity frightening. An inexpensive, non-motorized, easy-to-use, painless, safe device for removing an applied cast on a body member of a patient while avoiding any injury to the underlying skin of the patient is needed.

Additionally, current methods for removing orthopedic casts have also dictated how casts are applied on the body. In current methods, layers of natural cotton or synthetic nylon padding are placed below the layer of the cast and above the skin of the body member. The padding serves two purposes. One is to prevent irritation of the skin by the overlying cast, a point that is particularly relevant with respect to the use of plaster of Paris, a natural product that has dust particles in its dry state. The other purpose is to prevent accidental abrasion of the skin by the rough and sometimes friction-induced heated edges of a cast saw blade during cast removal. Further, application of padding under the cast is disadvantageous because cast material, e.g., plaster of Paris, cannot be closely applied to the body in the presence of padding. The cast removal device described herein will minimize the need to use cast padding in all instances of cast applications, leaving its use for application to parts of the body that have bony prominences or for protecting wound surfaces under a cast.

### SUMMARY OF THE INVENTION

Methods of applying a cast removal device to a patient, the east removal device, and methods of removing a cast described herein have the advantage over prior art devices and methods by minimizing or eliminating the use of padding under the cast. The molding of an applied cast to a body member, such as a fractured extremity becomes more fitting and conforming on the body without layers of padding and achieves better fracture alignment.

In one aspect, the invention is directed to a cast removal device comprising a cord having an abrasive portion, a first end, a second end, and a length, and a housing, wherein said housing features an elongated trough having a length and sized to house said elongated cord. The elongated cord has a length exceeding the length of the housing. In a particular embodiment, the elongated cord of the cast removal device may be substantially linear at rest, zig-zag shaped at rest or coiled at rest. The resting, zig-zag, or coiled cord is extendable to a substantially linear shape.

In another embodiment of the invention, the cast removal device includes at least one grip handle. The grip handle may be fixedly or detachably secured to one of the ends of the elongated cord.

The cast removal device may further comprise a semi-rigid tube having a longitudinal lumen paralleling the long axis of the semi-rigid tube and a length. The length of the semi-rigid tube is substantially the same length as the housing. The semi-rigid tube is positioned within the elongated trough of the housing. In one embodiment of the invention, the elongated cord is positioned within the semi-rigid tube.

Optionally, with respect to the cast removal device, the lumen of the semi-rigid tube has a removable material substantially filling the lumen from a first end of the semi-rigid tube to a second end of said semi-rigid tube.

In one embodiment, the housing has a flat, non-troughed portion positioned at each of the first and second end of the housing. The flat non-troughed portion in another embodiment includes an angled portion extending from the flat portion to the housing at an angle relative to the longitudinal axis of the housing of no less than 120 degrees.

In another aspect, the invention is directed to a method for removing a cast from a patient's body member using the cast removal device described herein. According to one embodiment of the method of cast removal, a healthcare provider applies tension to the elongated cord positioned in the housing having an elongated trough extending within the long axis of the housing. The housing is pre-positioned under the cast and over the skin of the body member. When the cast is to be removed from its position on a body member, the elongated cord is reciprocated to and fro cutting through the housing and the cast. The cast is removed from the body member. The ends of the elongated cord may be coupled to a grip handle such as a hook or loop and the grip handle is grasped by a healthcare provider for manually reciprocating the elongated cord to cut the cast.

In another embodiment, the method further includes introducing the elongated cord within the elongated trough.

Alternatively, the method further includes introducing a semi-rigid tube and removing the semi-rigid tube before reciprocating the elongated cord. The semi-rigid tube features a compressible material that substantially fills the lumen of the semi-rigid tube.

In one embodiment, the method of removing a cast using the cast removal device described herein includes introducing a semi-rigid tube into the housing, removing the semi-rigid tube, and introducing the elongated cord into the cannula space under the cast left by the semi-rigid tube.

In yet another aspect, the invention is directed to a method for applying a removable cast to a body member. The method includes providing a cast removal device having an elongated abrasive cord and a housing, placing the elongated cord and the housing above the skin of the body member, and applying the cast over the housing.

In another embodiment of the method, the elongated cord is pre-positioned in the housing before applying the housing to the body member. The method may include positioning the elongated cord in the housing after the housing is placed above the skin of the body member. Alternatively, the method includes positioning a semi-rigid tube in the housing before the elongated cord is positioned in the housing, and, optionally, removing the semi-rigid tube from the housing followed by introducing the elongated cord into the housing.

### BRIEF DESCRIPTION OF THE FIGURES

The above-mentioned objectives and advantages of the present invention will become more apparent and the invention itself will be best understood by reference to the following description of the present invention taken in conjunction with the accompanying drawings. The drawings are not to scale and emphasis is generally placed upon illustrating the principles of the invention.

FIG. 1A illustrates a perspective view of one embodiment of the cast removal device according to the invention with a first and second elongated cord in position deep to a cast on a lower extremity;

FIG. 1B illustrates an exemplary straight elongated cord, an exemplary coiled elongated cord and an exemplary zig-zag elongated cord, each at rest according to an embodiment of the invention.

FIG. 1C illustrates the elongated cords illustrated in FIG. 1B in an extended, non-rested position according to an embodiment of the invention.

FIG. 2 illustrates a plan view of an elongated cord and a handle grip at each end of the elongated cord according to one embodiment of the invention;

FIG. 3A illustrates a top view of an exemplary cast removal device including a housing, elongated trough and an elongated cord according to an embodiment of the invention;

FIG. 3B illustrates a perspective view of one end of the cast removal device illustrated in FIG. 3A taken at line 3B-3B;

FIG. 4A illustrates a transverse section of the embodiment of the cast removal device applied to a body member according to the embodiment of the invention illustrated in FIG. 1A at line 4A-4A;

FIG. 4B is an enlarged view of a transverse section of a portion of the cast removal device illustrated in FIG. 4A.

FIG. 5 illustrates an exemplary housing of the cast removal device positioned deep to the cast and having flattened terminal ends extending at an angle from the housing and resting on the exterior surface of the cast according to one embodiment of the invention.

FIG. 6A illustrates a transverse section of an exemplary cast removal device including a housing and a semi-rigid tube according to an embodiment of the invention.

FIGS. 6B-6D illustrate perspective views of the exemplary cast removal device shown in FIG. 6A in various stages of assembly according to an embodiment of the invention.

FIG. 7 illustrates an exemplary retrieval device for retrieving the ends of the elongated cord while the elongated cord is positioned under the cast according to one embodiment of the invention.

FIG. 8A illustrates one embodiment of the cast removal device positioned in the web space between the fingers of the hand according to one embodiment of the invention.

FIG. 8B illustrates one embodiment of a retrieval device for introducing and removing the elongated cord positioned under a cast adjacent to the toes or fingers.

FIG. 9A illustrates an exemplary cast removal device formed by a plurality of overlapping housings according to an embodiment of the invention.

FIG. 9B illustrates a single-unit frame including four fixedly joined housings according to another embodiment of the invention.

### DETAILED DESCRIPTION OF THE INVENTION

All of the following embodiments of the cast removing device described herein generally have at least one thing in common, namely, an elongated cord and a housing and/or, alternatively, an elongated cord, a housing, and a semi-rigid tube for the elongated cord.

The present invention provides devices, kits and methods for removing an orthopedic cast from a body part (hereinafter “body member”), of a patient. As used herein, a patient is a mammal, for example, a human. Body members include, but are not limited to, an arm, elbow, wrist, hand, finger, shoulder, leg, hip, pelvis, knee, ankle, foot, toe, trunk, neck, head, back, spine, clavicle, and joints. Specifically the present invention is directed to devices and kits for removing an orthopedic cast made of cast materials such as but not limited to plaster of Paris, polyurethane, fiber glass, other polymers, or combinations thereof applied to a body member typically to stabilize the body member to permit healing. Additionally, the invention is directed to applying and manufacturing such cast removal devices and kits.

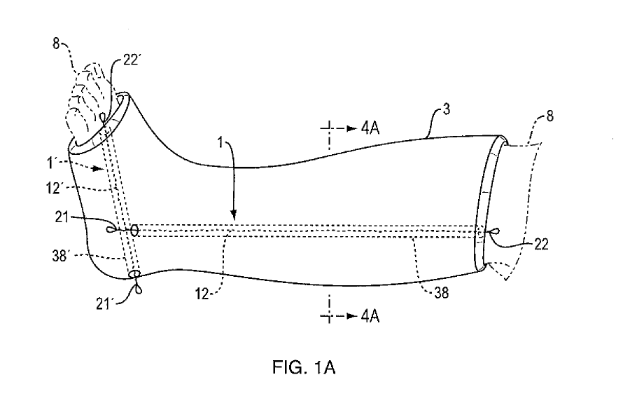

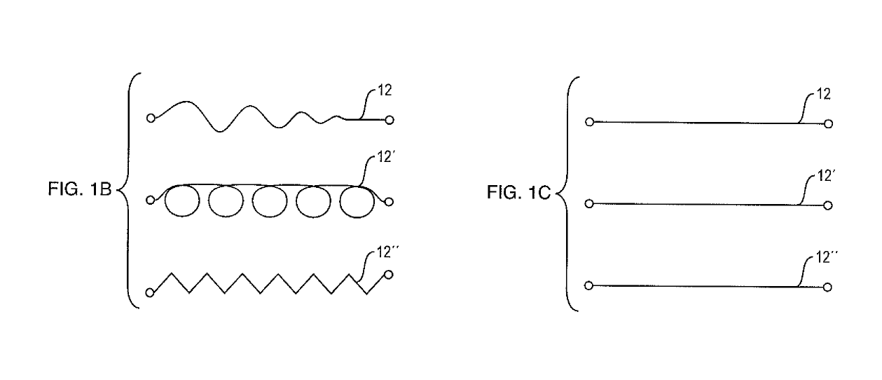

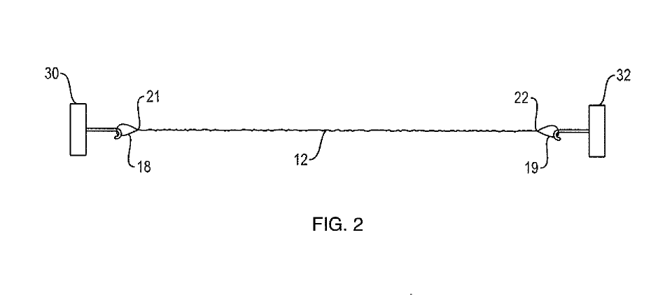

In one aspect, referring generally to FIG. 1A, a cast removal device 1 of the invention includes at least one cord 12 and a housing 38. Alternatively, a plurality of cast removal devices, 1, 1′ may be applied to a body member. The cord 12 typically is elongated, having at least two free ends 21, 22, and, alternatively, attachment devices 18, 19 at the free ends 21, 22 to releasably connect each of the free ends 21, 22, respectively, of the elongated cord 12 to one of the grip handles 30, 32 illustrated in FIG. 2. In one embodiment, referring to FIG. 1B, at rest the cord 12 comprises a substantially straight element, a looped element, a coil 12′, or a zigzag 12″. Each of the cords 12, 12; and 12″ may be transitioned to a linear extended form illustrated in FIG. 1C. As used herein, elongated cord refers to a cord that is substantially linear at rest or transitionable to a substantially linear state when not at rest.

The entire length, or, alternatively, portions of the length of cord 12 are abrasive and are made out of or covered with abrasive materials such as, but not limited to, metallic material or composites such as plastics with similar tensile strength to metal, abrasive carbon reinforced steel wire, electropolished steel wire, non-electropolished steel wire, Teflon®-coated abrasive wire, Dicronite coated wire, stainless steel, titanium, or ion nitride.

In a particular embodiment of the invention, the free ends 21, 22 of the elongated cord 12 are made of a material that is non-abrasive relative to the abrasive portion of the elongated cord 12.

In a particular embodiment of the invention, the abrasive cord 12 is lubricated and textured so that it slides and cuts easily through the casting material. Lubricants are selected from the group consisting of but are not limited to Dicronite™, or Teflon®, similar liquid or solid lubricants, for example, oils, petroleum jelly, or wax, or combinations thereof. In one embodiment, the elongated cord 12 is hardened by an abrasive diamond flurry coating to improve its cast material cutting properties. The elongated cord may be textured to provide a roughened or sharp abrasive cutting surface. Lubricants may be placed over the diamond flurry or the roughened, sharp abrasive surface to ease cutting of cast materials by the cord.

As illustrated in FIG. 2, in one embodiment of the invention, each of the free ends 21, 22 of the abrasive elongated cord 12 have attachment devices 18, 19, respectively, to connect in a releasable manner to one of a pair of grip handles 30, 32, respectively. Alternatively, during manufacture the grip handles 30, 32 are incorporated into the ends 21, 22 of the elongated cord 12 to be deployed during use, such that there is no requirement for handles to be coupled to the ends of the elongated cord at the time the cast is cut by the elongated cord 12.

In one embodiment, the attachment devices 18, 19 are threaded female members for coupling with threaded male members, respectively, on the grip handle 30, 32. Alternatively the threaded female member is positioned on the grip handle and the threaded male member is positioned on the attachment devices 18, 19 of the abrasive cord 12.

In yet another embodiment, the attachment devices 18, 19 and the grip handles 30, 32 are “snap-locked” together.

In still another embodiment for joining one end of the elongated cord 12 to a grip handle, the end of the elongated cord 12 comprises a loop attachment device 18 as illustrated in FIG. 2 that may be connected to a loop or hook component of a grip handle 30. Alternatively, each of the free ends 21, 22 of the elongated cord 12 are fitted into one of the grip handles 30, 32 and crimped into position with a crimping device.

In still another embodiment of the invention, the limb of a T-handle having only one free end has a cannulated central bore. The cannulated central bore receives a free end 21, or 22 of the elongated cord 12 which is then securely locked in the central bore with a locking screw positioned perpendicular to the long axis of the inserted elongated cord 12.

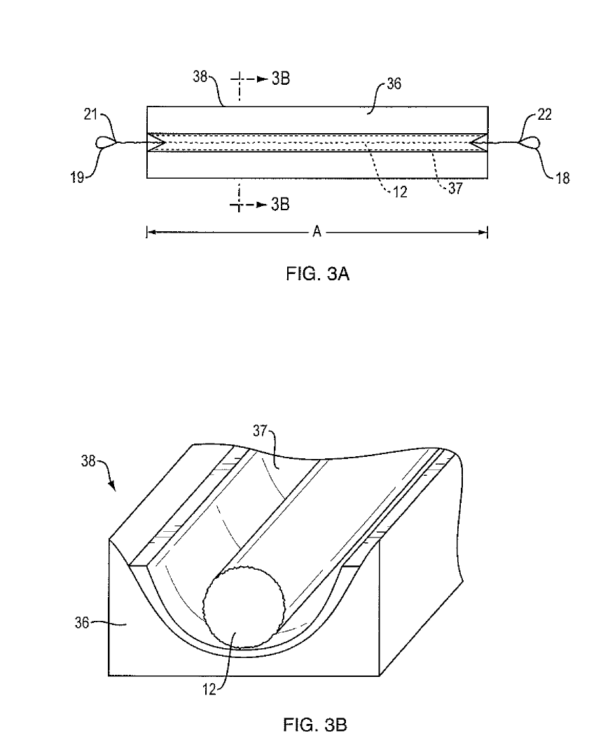

Referring to FIGS. 3A and 3B, in another embodiment of the invention, the housing 38 comprises a panel 36. The panel 36 is narrow, has a long axis A and extends parallel to the long axis of the cast in the range of about 50-75%, 50-85%, 75-100%, 100% of the length of the cast or, alternatively, 10-50% longer than the length of the cast (not shown). Referring to FIG. 3B, in a preferred embodiment, the panel 36 comprises a grooved channel 37 that extends parallel to the long axis of the panel 36 for about the same length as the long axis of the panel 36.

In one embodiment, the channel 37 is tube-like that is open along at least a portion of its length, e.g., a trough shape forming a half round trough, or quarter round trough, for example, as illustrated in FIG. 3B. The radius of the channel 37 is in the range of about 1/32 to 1.5 inches, 1/32 to 1.0 inches, or preferably about 1/16 to 1 inches.

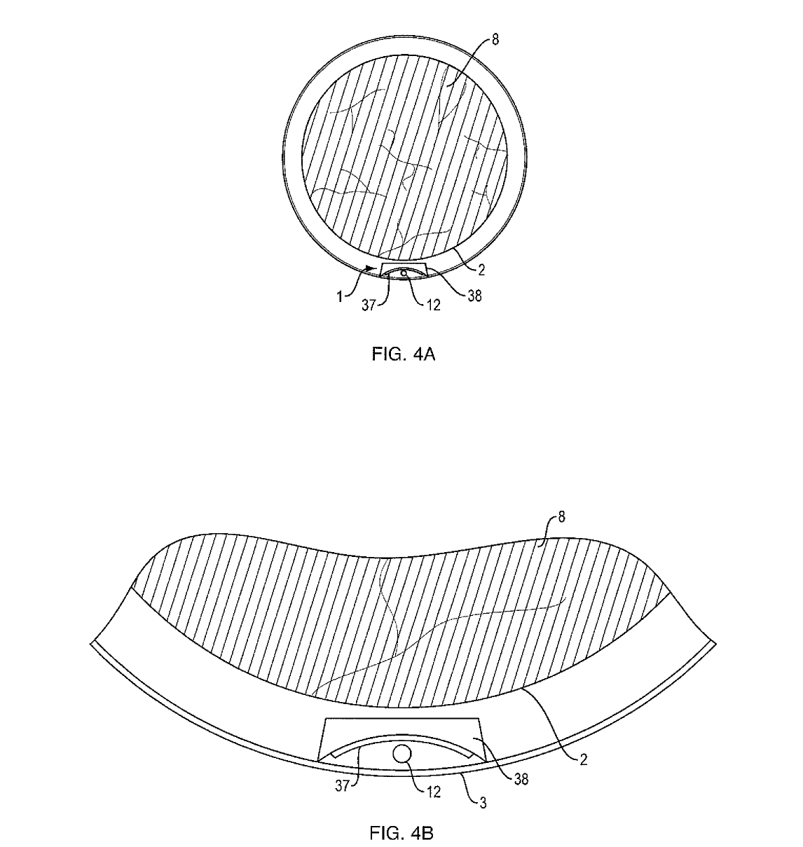

Referring now to FIGS. 4A and 4B, the housing 38 is placed above the skin 2, i.e., superficial to the skin and deep to the cast 3 when the cast material is being applied to the body member 8. The housing 38 serves as a protective shield for the skin 2 while healing of the body member 8 takes place with the cast 3 applied to the body member 8.

With further reference to FIGS. 4A and 4B, the channel 37 of the housing 38 is positioned on the side away from the skin 2, i.e., on the side of the cast material 3. The radius of the channel 37 is sufficiently large to receive the elongated cord 12 while simultaneously providing adequate room for the cord 12 to be pulled freely while deep to the layers of casting materials.

The housing 38 is preferably made of a translucent material, for example, but not limited to plastic composite or a mixture of multiple materials for the different parts so as not to obstruct clarity of the underlying bone of the body member during imaging studies. The wall of the housing 38 is preferably thin, for example, 1/64 to 1/16 inches, 1/64 to 1/32 inches, preferably 1/32 inches thick such that the housing will fit underneath an applied cast without exerting undue pressure on the skin of the patient.

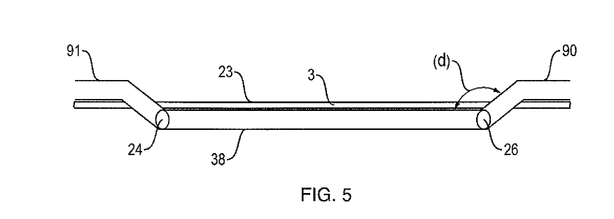

Referring to FIG. 5, the housing 38 has ends 24, 26. In one embodiment of the invention, the ends 24, 26 of the housing 38 depart at an angle (d) of about no less than 120 degrees, 120 to 165 degrees, or 150 to 165 degrees as measured from the longitudinal axis of the housing 38. The housing ends 24 and 26 may be flattened 91,90, respectively, to rest parallel with and flush against the exterior surface 23 of the applied cast 3.

In a particular embodiment, the housing 38 has its ends 24, 26 configured into a slit tubular end that connects to the exterior of an applied cast enabling the elongated cord to exit when tension is applied to remove the overlying cast.

The different configurations of the housing 38 of the cast removal device 1 of this invention can also be dispensed in a continuous roll from a pack such that the required length is pulled out and trimmed once the optimum length is determined. The elongated cord can also be packaged in the same manner—in a roll such that the optimum length can be dispensed at the time of use.

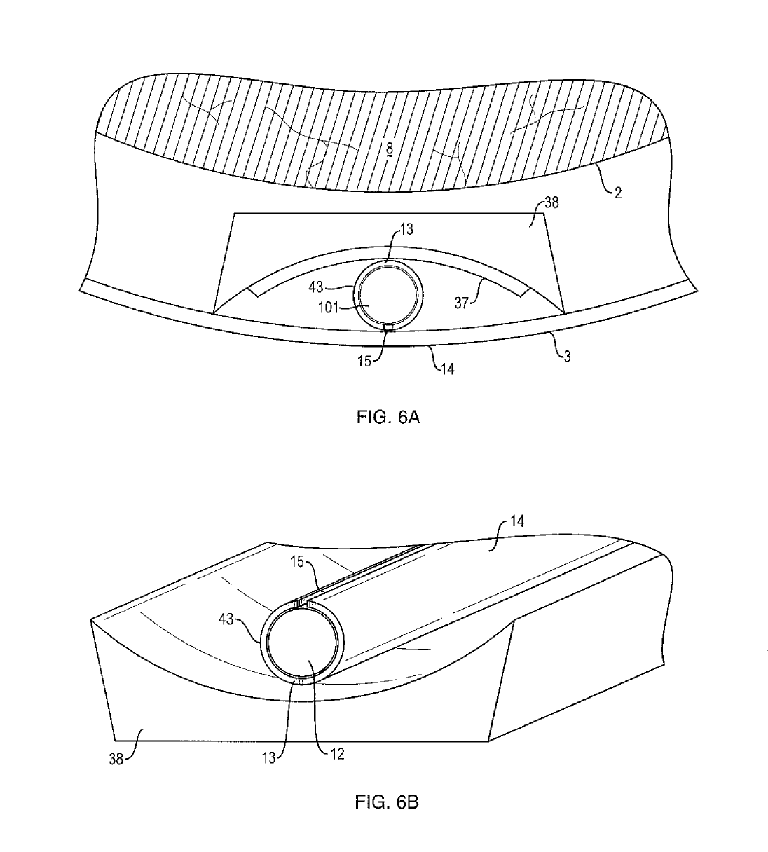

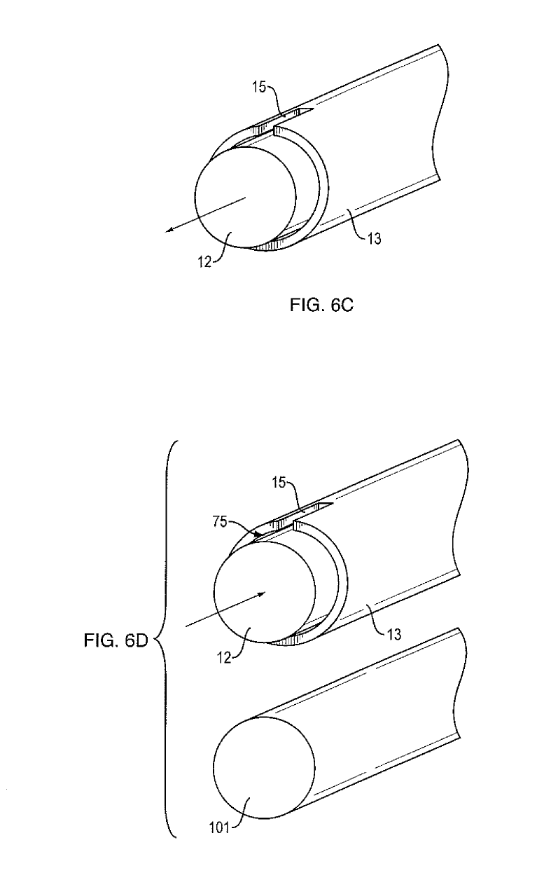

Referring to FIGS. 6A to 6D, in another embodiment of the invention, the housing 38, is fitted with an elongated hollow semi-rigid tube 13 that is removed after cast application. The cannulated channel 75 that remains after the semi-rigid tube 13 is removed receives the elongated cord 12 aiding the passage of the elongated cord from one end of the housing 38 to the other end of the housing 38. In one embodiment of the invention, the semi-rigid tube 13 has one or both ends configured into a slit 15 enabling the elongated cord 12 to exit when pulled on to remove an overlying cast. The elongated cord 12 may be pre-placed in the tube 13 of the housing 38 before the cast is applied to a body member. Alternatively, the elongated cord 12 may be inserted into the tube 13 after the cast removal device is introduced under the cast at the time the cast is applied to the body member.

The semi-rigid tube 13 may be made of a preferably translucent semi-rigid plastic or any suitable semi-rigid, flexible material. The tube 13 may be perforated (not shown) or include a slit 15 through the wall 43 of the tube 13 along all or part of its length to allow for easy egress of the elongated cord 12 of the cast removal device 1 when the cord is readied to cut the cast material.

The semi-rigid 13 tube is positioned with the housing 38 in the patient while the cast material is being applied and may be left in place or may be removed after the application of the casting material on the patient's body member.

In one embodiment, the surface 43 of the semi-rigid tube 13 is lubricated with Teflon®, wax, or oil to permit its easy withdrawal from under the cast while covered with layers of casting material. Alternatively, lubrication can be achieved by applying a layer or liner of polyethylene material around the tube 13 before it is covered with layers of casting materials. In one embodiment of the invention, the elongated cord 12 is pulled inward into the housing 38 or the tube 13 by a pre-positioned cord, suture, wire, or thread manufactured into the housing or tube.

In yet another embodiment of the invention, the semi-rigid tube 13 is collapsible. Referring to FIG. 6A, a removable central rod 101 extends in the lumen of the tube 13 from one end of the tube 13 to the other end of the tube to prevent the tube from collapsing when layers of casting materials are wrapped over it. The rod 101 is made from a soft, compressible, semi-rigid material, for example, but not limited to silicone, vinyl, and rubber. The rod 101 is semi-rigid and slightly bendable from about 2° to 20° measured from the long axis of the rod. The rod 101 largely fills the lumen of the tube 13 as illustrated in FIGS. 6A and 6C.

In one embodiment of the invention, the rod 101, as part of the cast removal device 1, is placed in the lumen of the semi-rigid tube 13 before the casting materials are applied. The rod 101 is removed (arrow of FIG. 6C) after the cast is set leaving a patent channel 75 for insertion of the elongated cord 12 as illustrated in FIG. 6D. The ends of the semi-rigid tube preferably extend, for example, about ⅛ to ½ inches, ¼ to ½ inches, preferably ¼ inches beyond the exterior surface of the layer of casting materials to offer easy access for positioning the elongated cord 12.

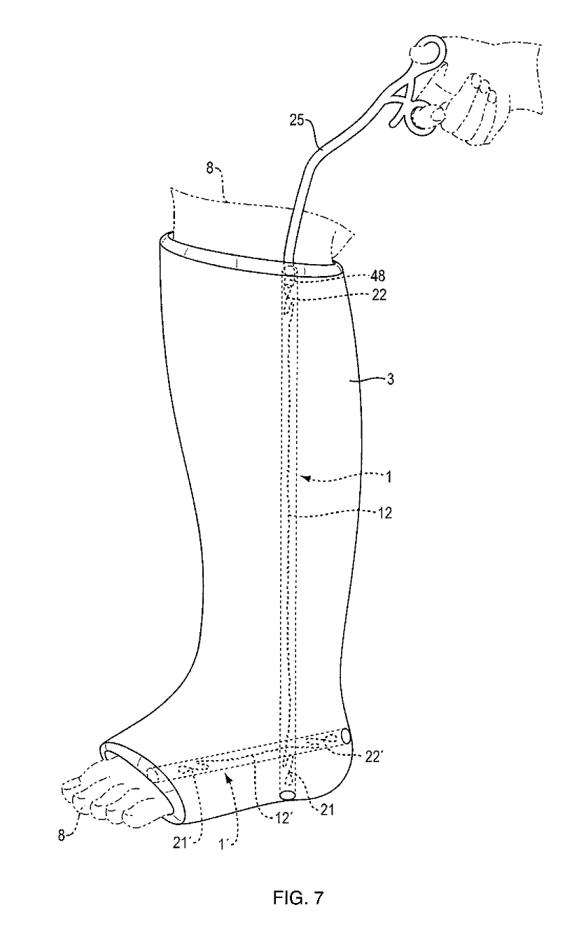

In one embodiment, referring to FIG. 7, an elongated cord 12 having a length that exceeds the length of the housing 8 is loosely positioned within the housing 8 with the ends 21, 22 of the elongated cord 12 enclosed with in the housing 8. A grip end 48 of a grasping device 25 similar to the grip end of a hemostat may be introduced into the housing 8 to grasp a free end 21 of the elongated cord 12 and pull out the free end 21. The process is repeated at the other end 22 of the elongated cord 12 to enable the healthcare provide to use the elongated cord 12 to cut the cast 3.

In instances when the available length of the elongated cord available is not long enough to extend fully through the length of the cast to be cut, one or more of the terminal ends of the cord may be connected to a non-abrasive cord or thread looped through the loop at the end of the elongated cord or by any other means, giving the cord the extra needed length. In such instance, one portion of the elongated cord will be effective in cutting through the cast while the non-abrasive part offers the length required to enable the abrasive cord to be reciprocated back and forth to cut the cast.

An alternative method for dealing with an elongated cord that is too short is to link up two short elongated cords at their ends using any connection means to yield a combined length adequate to extend through the length of the cast.

In the same manner that two or more elongated cords can be joined to one another to achieve a longer length, two or more housings can be affixed to one another to achieve a longer length, using connectors, for example, hooks or loops, or combinations thereof compatible with the cross-sectional shape of the housing.

In another adaptation of the current invention, the part of the housing 38 that abuts on the layers of cotton padding is coated with an adhesive material. Adhesive materials include but are not limited to epoxy, polyamide, and polyurethane. Adhesives assist in maintaining the housing 38 in position during the application of casting materials to a body member making the procedure less cumbersome. The housing 38 may be secured in place during application by a number of means, including the use of a layer of adhesive, an elasticated band, or taping. The semi-rigid tube 13 can replace the housing component during use and can be used in combination with the housing 38.

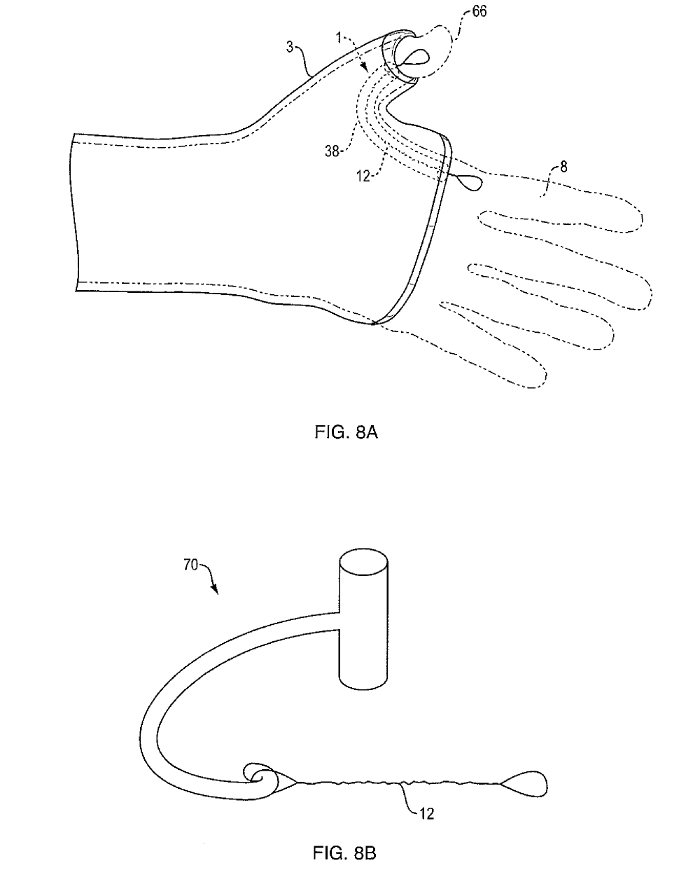

The cast removal device and method thereof described above is not limited to application to an extremity of the body. For example, as illustrated in FIG. 8A, the cast removing device 1 may be applied to the fingers of a hand 66 or to parts of a cast 3 that cover the wrist and extends over the thumb, or the toes of the foot (not shown), the cast extending over the toes and the ankle, for example. The cast removal device 1 according to the invention and described above may extend between the fingers or the toes in the web spaces of the hand or the foot, respectively, alternatively extending out from under the rim of the cast.

FIG. 8B illustrates a retrieval device 70 comprising an elongated member that is curved, a handle and a free end having an attachment device, e.g., a hook.

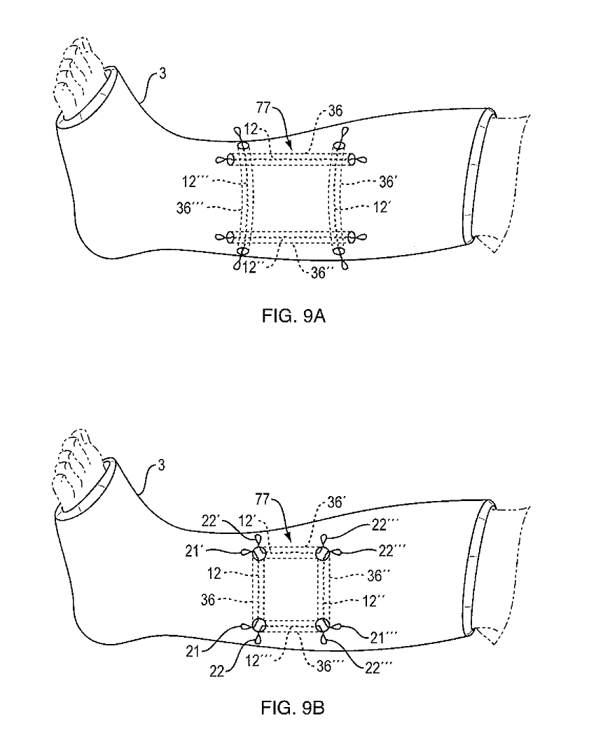

Referring to FIGS. 9A and 9B, in another embodiment of the cast removal device, a frame 77 shaped in the configuration of a circle, semi-circle, square, triangle, or any desired shape comprises, for example, multiple housings 36, 36′, 36″, 36′″ combined to form the specified shape, for example, the rectangular shape 77 shown in FIGS. 9A and 9B. As illustrated in FIG. 9B, in one embodiment, multiple housings are fixedly joined together to form a single unit frame in which a channel in each of the four housings 36, 36′, 36″, 36′″ is available to accept an elongated cord 12. Not illustrated in FIG. 9B, but a single elongated cord 12 could be used in one housing to the next housing and so on rather than an elongated cord 12 per housing 36. Alternatively, an elongated cord 12 is inserted into each of the housings 36 such that each end of an elongated cord 12 overlaps with one of the ends of another elongated cord 12 as shown in FIGS. 9A and 9B. Individual housings arranged under the cast 3 to form a shape such as the rectangle illustrated in FIG. 9A or the single units frame 77 are pre-applied above the skin before the cast material is applied to the patient's body member and remains in place under the cast material until an opening through the cast material is desired, for example, to provide a portal for introduction of a medication, or to provide an opening through cast material over a boney prominence where pressure sores may develop. The opening is made by applying outward tension to each end of the elongated cords 12 inserted in each of the housings 38 and reciprocatingly transitioning the elongated cord 12 to and fro. By abrasive cutting force applied by tension on the cord and reciprocating motion of the cord, a piece of the casting material roughly in the shape of the frame is removed.

In another aspect, the invention is directed to a method of applying the cast removal device described above to the body member of a patient. According to one embodiment, typically more than one cast removal device including the housing component 38 is positioned above the skin, for example, preferably on the skin, or above a thin liner applied to the skin under the cast, or, alternatively above (superficial to) a layer of protective padding on the skin or merely a thin nylon or other polymer liner that abuts the skin. For example, referring back to FIG. 1A, a cast removal device 1 may be positioned on opposite sides of the body member 8 or one or more cast removal devices may be applied at right angles to one or more cast removal devices 1′ on the same body member 8.

The elongated cord 12 may be pre-loaded into the housing 38 before the cast removal device 1 is applied to the body member 8. Alternatively, the housing 38 may be positioned on the body member 8 without a pre-loaded elongated cord 12. In another embodiment the elongated cord 12 may be pre-loaded into the semi-rigid tube 13. When an elongated cord 12 is loaded into its housing 38 for use, the elongated cord 12 is preferably at least one and a half times the length of the cast 3 in order to give a degree of slack for a user to handle when reciprocating the elongated cord 12 to and fro to cut the cast 3. In one embodiment, the grip handles 30, 32 are pre-attached to the free ends 21, 22 of the elongated cord 12.

In an embodiment in which the housing 38 comprises flattened terminal ends 90 and 91, as illustrated in FIG. 5, the housing 38 is positioned as above, the cast material 3 is applied over the housing 38 and the terminal flattened ends 90 and 91 of the housing 38 are avoided while the cast 3 is being applied to avoid burying the terminal flat ends 90, 91 in the cast 3.

In another aspect, the invention is directed to removing a cast 3 from the body member 8 of a patient. According to one embodiment of this aspect of the invention, a pair of handles 30, 32 adapted to be easily gripped by the hands of a healthcare provider such as a physician or physician's assistant, are pre-applied to the elongated wire 12 of the cast removal device 1 prior to application of the cast 3 or, alternatively, are fitted to the terminal portion abutting the free 21, 22 of the elongated cord just before cast removal, in other words, after the cast has been applied to the body member. The healthcare provider, grasps the handles 30, 32 and using a reciprocating to and fro motion, tension is applied to the ends of the elongated cord 12 pulling it to and fro with outward pressure reciprocatingly until the elongated cord 12 exits the housing 38 and the cast 3 by cutting through the overlying casting material. This process of cast removal is repeated on at least two sides of the applied cast, using the pre-positioned cast removal devices that are pre-positioned above the skin but under the cast material when the cast is applied to the body member. In a long extremity of an adult, it may be necessary to remove a small segment of an applied cast at a time, rather than removing the whole length of the cast at once. In such situations, more than one unit of the cast removal device may be required on one side of the cast, with the cast removal devices positioned in series, or end-to-end along the length of an applied cast, and/or on two or more sides of an applied cast. When the cast is applied to the lower extremity, a separate unit of the cast removal device may be required beyond the level of the ankle joint with a degree of overlap over the first unit of the cast removal device so that the whole length of the cast may be fully cut from the leg to the foot.

With respect to the method of use, the limb encircled with a cast should be positioned on a suitable stand such that a steady counter-traction may be applied on the limb keeping it steady and preventing it from being repeatedly pulled as the elongated cord of the current invention is pulled in an opposite direction

With respect to the method of use, even though the original method of use of the cast removal kit requires that two hands pull on the handles connected to the ends of the elongated cord, other modification of use are possible including attaching one of the two ends of the elongated cord to one or more fixed handles and moving the limb encircled with a cast to and fro over the elongated cord.

In another embodiment of the invention, one or more parts of the elongated cord 12 may be reversibly coupled to a motorized device operated by battery, solar, or electric power for reciprocating the elongated cord 12 to and fro. In such instances, the power required is much less than a motorized oscillating saw as the power of the motor of the coupled machine is diverted towards the tensioning of the coupled abrasive wire as opposed to that of an oscillating saw. The coupled motorized machine is also less noisy as the motor power required is minimal.

Variations, modifications, and other implementations of what is described herein will occur to those of ordinary skill in the art without departing from the scope and spirit of the invention as claimed. Accordingly, the invention is to be defined not by the preceding illustrative description but instead by the scope and spirit of the following claims.

The invention claimed is:

1. A cast removal device comprising:
a cord comprising an abrasive portion, a first end, a second end, and a length; and,
a housing wherein said housing comprises an elongated trough having a length and sized to house said cord, said cord comprising a length exceeding the length of said housing.

2. The cast removal device of claim 1 further comprising a semi-rigid tube comprising a lumen, a first end, a second end and a length substantially the same length of said housing and positioned within said elongated trough, said cord positioned within said semi-rigid tube.

3. The cast removal device of claim 2 wherein the lumen of said semi-rigid tube comprises a removable material substantially filling said lumen from the first end of said semi-rigid tube to a second end of said semi-rigid tube.

4. The cast removal device of claim 1 wherein said housing comprises a flat, non-troughed portion positioned at each of said first end and second end of said housing.

5. The cast removal device of claim 4 wherein said flat non-troughed portion further comprises an angled portion extending from said flat non-troughed portion to said housing at an angle relative to a longitudinal axis of said housing no less than 120 degrees.

6. The cast removal device of claim 1 wherein said cord is zig-zag shaped at rest and extendable to a substantially linear shape.

7. The cast removal device of claim 1 wherein said cord is coiled at rest and extendable to a substantially linear shape.

8. The cast removal device of claim 1 further comprising at least one grip handle, said at least one grip handle fixedly secured to one of said first and second ends of said cord.

9. A method for removing a cast from a patient's body member, comprising:
applying tension to an elongated cord positioned in a housing, said housing comprising an elongated trough, said housing positioned under said cast and over the skin of said patient's body member;
reciprocating said elongated cord to and fro;
removing said cast from said patient's body member.

10. The method of claim 9 further comprising introducing said elongated cord within said elongated trough.

11. The method of claim 9 further comprising a semi-rigid tube having a lumen positioned in said trough and removing said semi-rigid tube before reciprocating said elongated cord.

12. The method of claim 9 further comprising,
introducing a semi-rigid tube into said housing;
forming a channel under said cast by removing said semi-rigid tube; and
introducing said elongated cord into said channel under said cast.

13. The method of claim 11 wherein the lumen of said semi-rigid tube comprises a compressible material.

14. The method of claim 9 further comprising coupling each of said first end and said second end of said elongated cord to a grip handle.

15. The cast removal device of claim 1 further comprising a grip comprising a hook.

16. The cast removal device of claim 1 further comprising a grip comprising a loop.

17. A method for applying a removable cast to a body member, comprising:
providing an elongated cord comprising an abrasive;
providing a housing;
placing said elongated cord and said housing above the skin of said body member;
applying said removable cast over said housing.

18. The method of claim 17 wherein said elongated cord is pre-positioned in said housing before applying said housing to said body member.

19. The method of claim 17 further comprising positioning said elongated cord in said housing after said housing is placed above the skin of the body member.

20. The method of claim 17 further comprising positioning a semi-rigid tube in said housing before said elongated cord is positioned in said housing.

21. The method of claim 20 further comprising removing said semi-rigid tube from said housing followed by introducing said elongated cord into said housing.
